(19) United States
(12) Patent Application Publication
(10) Pub. No.: US 2025/0127147 A1
(43) Pub. Date: Apr. 24, 2025

(54) Smart Collar Device

(71) Applicant: Renee Gibson, Casper, WY (US)

(72) Inventor: Renee Gibson, Casper, WY (US)

(21) Appl. No.: 18/914,385

(22) Filed: Oct. 14, 2024

Related U.S. Application Data
(60) Provisional application No. 63/592,615, filed on Oct. 24, 2023.

(51) Int. Cl. A01K 27/00; A01K 11/00; A01K 29/00; A61B 5/00; A61B 5/0205; A61B 5/11

(52) U.S. Cl. CPC A01K 27/009; A01K 11/008; A01K 27/001; A01K 27/006; A01K 29/005; A61B 5/0022; A61B 5/0205; A61B 5/1112; A61B 5/6802; A61B 5/7445

(57) ABSTRACT

A smart collar device is disclosed that is a durable pet collar that can be attached to a body harness or used individually. The smart collar device has electronic capabilities that monitor stress levels, oxygen levels, blood pressure, and heart rate. The smart collar may also include LED lights, GPS tracker, a camera and reflective straps. The smart collar can connect to an app to provide pet owners with data and reminders. The smart collar may be available in a wide variety of colors and sizes.

### CROSS-REFERENCE TO RELATED APPLICATION

The present application claims priority to, and the benefit of, U.S. Provisional Application No. 63/592,615, which was filed on Oct. 24, 2023, and is incorporated herein by reference in its entirety.

### FIELD OF THE INVENTION

The present invention relates generally to the field of smart collar devices. More specifically, the present invention relates to a smart collar for pets designed to monitor high blood pressure, heart rate, stress or anxiety levels. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in smart collar devices. Generally, pets suffer from stress, anxiety, low oxygen levels, and high blood pressure just like humans do, however, there are no reliable indicators. Further, pets may be in distress and are unable to alert their owners. Thus, without medical equipment from the veterinarian, these issues go unnoticed.

Generally, our society has become more interested in utilizing technology to assist with making our pet lives easier, healthier, and more fun/interactive. Further, to ensure safety of a domesticated house pet such as a dog and to prevent the pet from becoming lost, most owners may desire a way to track their pets and monitor their wellbeing at all times. However, conventional collars do not provide this functionality. For example, pet owners may have difficulty trying to find a location tracker that can be attached to a dog or other pet. It would be convenient to have a device that can be worn by a dog or other pet without any discomfort and which is able to track the location of the dog or other pet, as well as monitor its wellbeing.

Accordingly, there is a demand for an improved smart collar device that allows users to monitor the wellbeing of their pets. More particularly, there is a demand for a smart collar device that monitors a pet's high blood pressure, heart rate, stress or anxiety levels.

Therefore, there exists a long felt need in the art for a smart collar device that provides users with a smart collar for pets designed to monitor high blood pressure, heart rate, stress, or anxiety levels. There is also a long felt need in the art for a smart collar device that communicates with a pet owner's watch or phone using an app to send food and medication reminders. Further, there is a long felt need in the art for a smart collar device that includes a GPS tracker and camera to allow pet owners to see a real time location of their pet. Moreover, there is a long felt need in the art for a device that offers LED lights for night walks and safety purposes. Further, there is a long felt need in the art for a smart collar device that ensures that pet owners can monitor the wellbeing of their pet at all times. Finally, there is a long felt need in the art for a smart collar device that is available in different colors and sizes.

The subject matter disclosed and claimed herein, in one embodiment thereof, comprises a smart collar device. The device is a durable pet collar that can be attached to a body harness or used individually. The smart collar device has electronic capabilities that monitor stress levels, oxygen levels, blood pressure, and heart rate. The smart collar may also include LED lights, GPS tracker, a camera and reflective straps. The smart collar can connect to an app to provide pet owners with data and reminders. The smart collar may be available in a wide variety of colors and sizes.

In this manner, the smart collar device of the present invention accomplishes all of the forgoing objectives and provides users with a device that monitors the wellbeing of their pet at all times. The device is a smart collar with electronic capabilities. The device is available in different colors and sizes.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a smart collar device. The device is a durable pet collar that can be attached to a body harness or used individually. The smart collar device has electronic capabilities that monitor stress levels, oxygen levels, blood pressure, and heart rate. The smart collar may also include LED lights, GPS tracker, a camera and reflective strips. The smart collar can connect to a software application to provide pet owners with data and reminders. The smart collar may be available in a wide variety of colors and sizes.

In one embodiment, the smart collar device comprises a band for wrapping around the neck of a dog or other suitable pet. The smart collar device comprises a nylon or polyester band for wrapping around the neck of a dog or other pet and has an appearance similar to a conventional dog collar for comfortable wearing of the collar device. The elongated band is adapted to encircle the neck of a dog in a similar fashion as conventional dog collars known in the art. The band can be made of a slash-proof and soft material such as nylon webbing, polyester, hemp, or metal, etc., or any other suitable material as is known in the art.

In use, the collar device is secured around the neck of a dog via a buckle and a clip or which mate together and are integrated into the band. Further, the buckle may comprise a key-locking mechanism that locks the clip into the buckle when engaged. Additionally, any other type of fastening mechanism such as hook and loop, Velcro®, buckle, snap, etc., or any other mechanical fastener as is known in the art can be used for securing the ends of the band.

In one embodiment, the device includes a housing attached to the band or directly integrated within the band, which includes a plurality of electronic components for monitoring the pet wearing the smart collar device, wherein the components include a wireless communication module for establishing a wireless communication channel with a remote electronic device, a camera to capture an image around the collar, a location detector for detecting the current location of the collar, electronic capabilities to monitor the pet, LED lights and a display screen, and a USB charging port for recharging an internal battery. A control button and indicator lights are disposed on the housing and/or directly on the band to activate or deactivate the components of the housing/band. Specifically, the control button is used for activating or deactivating the electronic components and can be used for activation when the dog is taken for a walk. For power saving properties, the components can be deactivated when the collar device is not worn by the dog. Further, indicator lights can alert a pet owner that the device is turned on and fully powered, as well as that the internal battery is low and needs recharged/replaced.

In one embodiment, the smart collar device includes electronic capabilities that are able to monitor stress levels, oxygen levels, blood pressure, and heart rate of the pet wearing the collar. The electronic capabilities include various sensors and other components necessary to provide the capability of processing all information gathered by the components as described herein. This data can then be displayed on an LED display on the collar and sent to the pet owner on their paired smart device via a software application to be uploaded, analyzed, and recorded, as needed.

In one embodiment, the housing comprises a GPS tracker and camera which allows pet owners to see a real time location of their pet. Thus, the wireless communications module receives real time images and location information from the paired pet collar and displays the received real time images and location information on the user's paired smart device. Specifically, the smart collar device of the present invention has a built-in GPS chip that determines the location of the dog collar device and provides the location information to the dog owner using the wireless communication module. The location detector unit or built-in GPS chip provides positional information to track the dog wearing the dog collar device and is useful in cases when the dog wanders away. The smart collar device of the present invention also comprises a built-in camera. The camera captures still or moving images in an area in relative proximity to the collar device and provides the images to the dog owner using the wireless communications module. Further, the dog owner can also receive real time information from the dog wearing the collar device. The alert or notification shown on the software application may be in the form of a push notification, audio alert or a visual alert, etc., or any other suitable notification as is known in the art.

In one embodiment, the smart collar device is configured to connect wirelessly to a remote electronic device, such as a smart phone or other smart device. The smart collar device includes a wireless communication module for establishing a wireless communication channel with the remote electronic device. Monitored data, reminders, captured images and the current location of the smart collar device are transmitted to the remote electronic device using the wireless communication channel. Further, the wireless communication module is in the form of an integrated circuit or a microchip antenna and is configured to provide Bluetooth/Wi-Fi capabilities. The wireless communication module establishes a wireless communication channel with an electronic smart device.

In one embodiment, the smart collar device connects to the electronic smart device, such as smartphone, personal digital assistant, or any other such type of electronic/mobile device using a wireless communication medium such as Bluetooth, Wi-Fi or any other similar short range or long-range wireless technology as is known in the art. The smartphone device comprises an installed software application that receives information related to the pets, such as location, biometrics, such as stress levels, oxygen levels, blood pressure, and heart rate, etc., and other such type of information, reminders, captured images, and current locations, etc.

In yet another embodiment of the present invention, a computer-readable storage medium comprising instructions that are executed by a processor of an electronic device is disclosed. The instructions cause the processor of the electronic device to establish a wireless communication channel with the smart collar device using a wireless interface, to transmit data from the paired pet collar using the wireless communication channel to the user's smart device, and to receive real time images and location information from the paired pet collar, and to display the received real time images and location information on the user's paired smart device. Further, the dog owner can also receive real time information from the dog wearing the collar device. The alert or notification shown on the software application may be in the form of a push notification, audio alert or a visual alert, etc., or any other suitable notification as is known in the art.

In yet another embodiment of the present invention, the smart collar device establishes direct communication between a pet owner and the pet for monitoring the wellbeing of the pet at all times. Thus, the smart collar device communicates with a pet owner's smart watch or smart phone using an app to send food and medication reminders. Accordingly, the smart collar device connects to an app to provide pet owners with data and reminders, as needed. Specifically, the smart collar device allows the pet owner to easily activate and deactivate reminders for themselves for various activities, such as but not limited to, food and medication reminders, scheduling veterinary appointments, and/or brushing their pet's teeth, etc. Similarly, the app will allow the pet owner to record instances of performing any of these or similar actions, as well.

In one embodiment, the smart collar device comprises a plurality of LED lights which offers safety for night walks and other safety purposes for the pet.

In another embodiment, the smart collar device comprises a plurality of reflective strips positioned on the collar for safety purposes for the pet. The reflective strips ensure the safety collar and the pet wearing the collar can be seen during night walks and other low light conditions.

In one embodiment, the smart collar device is integrally formed into a collar or can be utilized as an aftermarket device and secured to a standard/off-the-shelf pet collar.

In one embodiment, the smart collar device will be water/dust resistant or waterproof as found suitable in the art in conditions normally experienced by pets in order to assure that the electronic capabilities will not lose functionality through normal daily use.

In one embodiment, the smart collar device of the present invention is durable, non-slippery, waterproof and scratch proof. Further, the smart collar device comprises an internal battery, which runs for up to forty hours or more. In one embodiment, the collar device may support built-in high-capacity lithium rechargeable batteries, however other suitable batteries may also be supported as is known in the art.

In one embodiment, the smart collar device also comprises a USB/mini-USB charging port which is used for recharging the internal battery that provides power to various components of the dog collar device.

In one embodiment, the smart collar device is available in a wide variety of colors, designs, patterns, and sizes.

In yet another embodiment, the smart collar device comprises a plurality of indicia.

In yet another embodiment, a method of monitoring the wellbeing of a pet via their collar is disclosed. The method includes the steps of providing a smart collar device comprising electronic capabilities. The method also comprises securing the smart collar device to a pet to be monitored. Further, the method comprises linking the smart collar device to a smart phone for data and reminders. The method also comprises monitoring the pet's stress levels, oxygen levels, blood pressure, and heart rate via the smart collar device. Finally, the method comprises allowing pet owners to see a real time location of their pet.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the smart collar device of the present invention showing the device worn on a pet in accordance with the disclosed architecture;

FIG. 2 illustrates a perspective view of one embodiment of the smart collar device of the present invention showing the health data being displayed in accordance with the disclosed architecture;

FIG. 3 illustrates a perspective view of one embodiment of the smart collar device of the present invention showing the smart camera in accordance with the disclosed architecture;

FIG. 4 illustrates a perspective view of one embodiment of the smart collar device of the present invention showing the device connected to a smart device in accordance with the disclosed architecture;

FIG. 5 illustrates a perspective view of one embodiment of the smart collar device of the present invention showing the device in use with a smart device in accordance with the disclosed architecture; and

FIG. 6 illustrates a flowchart showing the method of monitoring the wellbeing of a pet via their collar in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a smart collar device that provides users with a smart collar for pets designed to monitor high blood pressure, heart rate, stress, or anxiety levels. There is also a long felt need in the art for a smart collar device that communicates with a pet owner's watch or phone using an app to send food and medication reminders. Further, there is a long felt need in the art for a smart collar device that includes a GPS tracker and camera to allow pet owners to see a real time location of their pet. Moreover, there is a long felt need in the art for a device that offers LED lights for night walks and safety purposes. Further, there is a long felt need in the art for a smart collar device that ensures that pet owners can monitor the wellbeing of their pet at all times. Finally, there is a long felt need in the art for a smart collar device that is available in different colors and sizes.

The present invention, in one exemplary embodiment, is a novel smart collar device. The device is a durable pet collar that can be attached to a body harness or used individually. The smart collar device has electronic capabilities that monitor stress levels, oxygen levels, blood pressure, and heart rate. The smart collar may also include LED lights, GPS tracker, a camera and reflective straps. The smart collar can connect to an app to provide pet owners with data and reminders. The smart collar may be available in a wide variety of colors and sizes. The present invention also includes a novel method of monitoring the wellbeing of a pet via their collar. The method includes the steps of providing a smart collar device comprising electronic capabilities. The method also comprises securing the smart collar device to a pet to be monitored. Further, the method comprises linking the smart collar device to a smart phone for data and reminders. The method also comprises monitoring the pet's stress levels, oxygen levels, blood pressure, and heart rate via the smart collar device. Finally, the method comprises allowing pet owners to see a real time location of their pet.

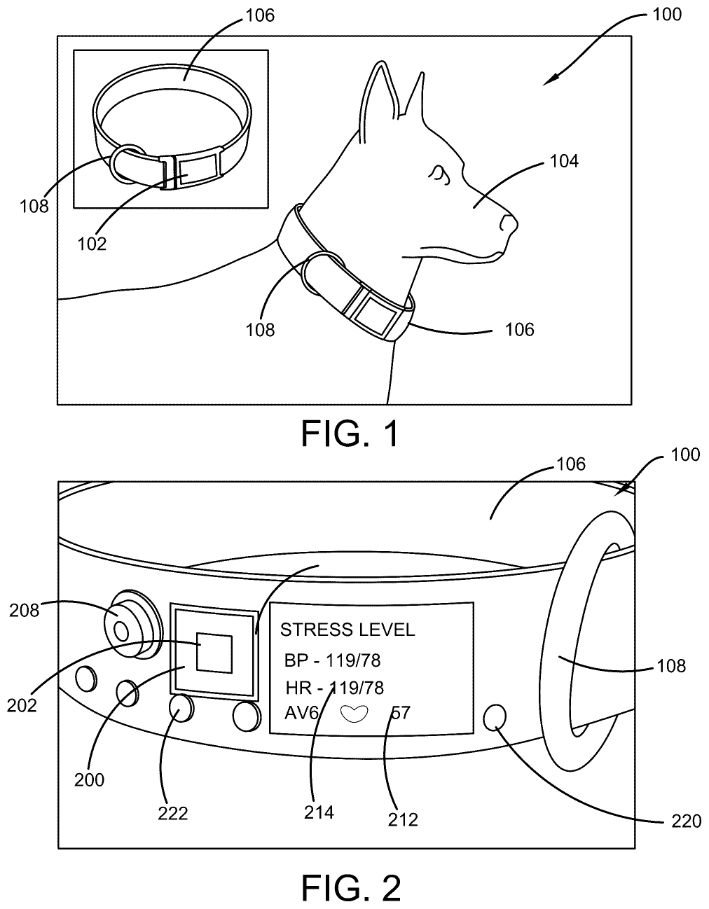

Referring initially to the drawings, FIG. 1 illustrates a perspective view of one embodiment of the smart collar device 100 of the present invention. In the present embodiment, the smart collar device 100 is an improved smart collar device 100 that provides a user with a means for monitoring the wellbeing of a pet. Specifically, the smart collar device 100 is a durable pet collar that can be attached to a body harness or used individually. The smart collar device 100 has electronic capabilities that monitor stress levels, oxygen levels, blood pressure, and heart rate. The smart collar 100 may also include LED lights, GPS tracker, a camera and reflective strips. The smart collar 100 can connect to a software application to provide pet owners with data and reminders.

Generally, the smart collar device 100 comprises a band 106 for wrapping around the neck of a dog 104 or other suitable pet. The smart collar device 100 comprises a nylon or polyester band 106 for wrapping around the neck of a dog 104 or other pet and has an appearance similar to a conventional dog collar for comfortable wearing of the collar device 100. The elongated band 106 is adapted to encircle the neck of a dog 104 in a similar fashion as conventional dog collars known in the art. The band 106 can be made of a slash-proof and soft material such as nylon webbing, polyester, hemp, or metal, etc., or any other suitable material as is known in the art.

In use, the collar device 100 is secured around the neck of a dog 104 via a buckle and a clip 108 or which mate together and are integrated into the band 106. Further, the buckle may comprise a key-locking mechanism that locks the clip into the buckle when engaged. Additionally, any other type of fastening mechanism such as hook and loop, Velcro®, buckle, snap, etc., or any other mechanical fastener as is known in the art can be used for securing the ends of the band 106.

As shown in FIG. 2, the device 100 includes a housing 102 attached to the band 106 or directly integrated within the band 106, which includes a plurality of electronic components 200 for monitoring the pet 104 wearing the smart collar device 100, wherein the components 200 include a wireless communication module 202 for establishing a wireless communication channel 204 with a remote electronic device 206, a camera 208 to capture an image around the collar 100, a location detector (i.e., GPS 210) for detecting the current location of the collar 100, electronic capabilities 200 to monitor the pet 104, LED lights 212 and a display screen 214, and a USB charging port 216 for recharging an internal battery 218. A control button 220 and indicator lights 222 are disposed on the housing 102 and/or directly on the band 106 to activate or deactivate the components of the housing 102/band 106. Specifically, the control button 220 is used for activating or deactivating the electronic components 200 and can be used for activation when the dog 104 is taken for a walk. For power saving properties, the components 200 can be deactivated when the collar device 100 is not worn by the dog 104. Further, indicator lights 222 can alert a pet owner that the device 100 is turned on and fully powered, as well as that the internal battery 218 is low and needs recharged/replaced.

Further, the smart collar device 100 includes electronic capabilities 200 that are able to monitor stress levels, oxygen levels, blood pressure, and heart rate of the pet 104 wearing the collar 100. The electronic capabilities 200 include various sensors and other components necessary to provide the capability of processing all information gathered by the components 200 as described herein. This data can then be displayed on an LED display 214 on the collar 100 and sent to the pet owner on their paired smart device 206 via a software application 224 to be uploaded, analyzed, and recorded, as needed.

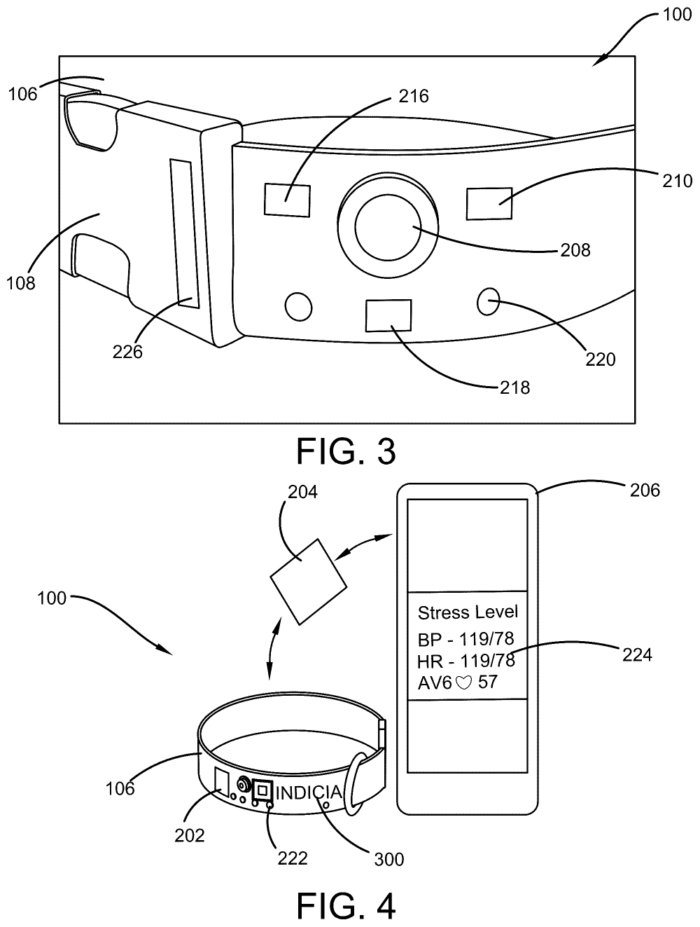

As shown in FIG. 3, the housing 102 comprises a GPS tracker 210 and camera 208 which allows pet owners to see a real time location of their pet 104. Thus, the wireless communications module 202 receives real time images and location information from the paired pet collar 100 and displays the received real time images and location information on the user's paired smart device 206. Specifically, the smart collar device 100 of the present invention has a built-in GPS chip 210 that determines the location of the dog collar device 100 and provides the location information to the dog owner using the wireless communication module 202. The location detector unit or built-in GPS chip 210 provides positional information to track the dog 104 wearing the dog collar device 100 and is useful in cases when the dog 104 wanders away. The smart collar device 100 of the present invention also comprises a built-in camera 208. The camera 208 captures still or moving images in an area in relative proximity to the collar device 100 and provides the images to the dog owner using the wireless communications module 202. Further, the dog owner can also receive real time information from the dog 104 wearing the collar device 100. The alert or notification shown on the software application 224 may be in the form of a push notification, audio alert or a visual alert, etc., or any other suitable notification as is known in the art.

Further, the smart collar device 100 comprises a plurality of LED lights 212 which offers safety for night walks and other safety purposes for the pet 104.

Additionally, the smart collar device 100 comprises a plurality of reflective strips 226 positioned on the collar 100 for safety purposes for the pet 104. The reflective strips 226 ensure the safety collar 100 and the pet 104 wearing the collar 100 can be seen during night walks and other low light conditions.

In one embodiment, the smart collar device 100 is integrally formed into a collar or can be utilized as an aftermarket device and secured to a standard/off-the-shelf pet collar.

Further, the smart collar device 100 will be water/dust resistant or waterproof as found suitable in the art in conditions normally experienced by pets 104 in order to assure that the electronic capabilities will not lose functionality through normal daily use.

Additionally, the smart collar device 100 of the present invention is durable, non-slippery, waterproof and scratch proof. Further, the smart collar device 100 comprises an internal battery 218, which runs for up to forty hours or more. In one embodiment, the collar device 100 may support built-in high-capacity lithium rechargeable batteries, however other suitable batteries may also be supported as is known in the art.

The battery 218 may be a disposable battery or a rechargeable battery in the form of an alkaline, nickel-cadmium, nickel-metal hydride battery, etc., such as any 3V-12 volts DC battery or other conventional battery, such as A, AA, AAA, etc., that supplies power to the smart collar device 100. Throughout this specification, the term “battery” may be used interchangeably to refer to one or more wet or dry cells or batteries of cells in which chemical energy is converted into electricity and used as a source of DC power. References to recharging or replacing the battery 218 may refer to recharging or replacing individual cells, individual batteries of cells, or a package of multiple battery cells as is appropriate for any given battery technology that may be used. In addition, a rechargeable embodiment of the battery 218 may be recharged using a USB port 216, wherein the USB port 216 is a USB-A, USB-B, Micro-B, Micro-USB, Mini-USB, or USB-C port, etc.

Further, the smart collar device 100 also comprises a USB/mini-USB charging port 216 which is used for recharging the internal battery 218 that provides power to various components of the dog collar device 100.

In yet another embodiment, the smart collar device 100 comprises a plurality of indicia 300. The housing 102 of the device 100 may include advertising, a trademark, or other letters, designs, or characters, printed, painted, stamped, or integrated into the housing 102, or any other indicia 300 as is known in the art. Specifically, any suitable indicia 300 as is known in the art can be included, such as but not limited to, patterns, logos, emblems, images, symbols, designs, letters, words, characters, animals, advertisements, brands, etc., that may or may not be collar, pet, or brand related.

As shown in FIG. 4, the smart collar device 100 is configured to connect wirelessly to a remote electronic device 206, such as a smart phone or other smart device. The smart collar device 100 includes a wireless communication module 202 for establishing a wireless communication channel 204 with the remote electronic device 206. Monitored data, reminders, captured images and the current location of the smart collar device 100 are transmitted to the remote electronic device 206 using the wireless communication channel 204. Further, the wireless communication module 202 is in the form of an integrated circuit or a microchip antenna and is configured to provide Bluetooth/Wi-Fi capabilities. The wireless communication module 202 establishes a wireless communication channel 204 with an electronic smart device 206.

Further, the smart collar device 100 connects to the electronic smart device 206, such as smartphone, personal digital assistant, or any other such type of electronic/mobile device using a wireless communication medium such as Bluetooth, Wi-Fi or any other similar short range or long-range wireless technology as is known in the art. The smartphone device 206 comprises an installed software application 224 that receives information related to the pets, such as location, biometrics, such as stress levels, oxygen levels, blood pressure, and heart rate, etc., and other such type of information, reminders, captured images, and current locations, etc.

In yet another embodiment of the present invention, a computer-readable storage medium comprising instructions that are executed by a processor of an electronic device 206 is disclosed. The instructions cause the processor of the electronic device 206 to establish a wireless communication channel 204 with the smart collar device 100 using a wireless interface, to transmit data from the paired pet collar 100 using the wireless communication channel 204 to the user's smart device 206, and to receive real time images and location information from the paired pet collar 100, and to display the received real time images and location information on the user's paired smart device 206. Further, the dog owner can also receive real time information from the dog 104 wearing the collar device 100. The alert or notification shown on the software application 224 may be in the form of a push notification, audio alert or a visual alert, etc., or any other suitable notification as is known in the art.

Further, the smart collar device 100 establishes direct communication between a pet owner and the pet 104 for monitoring the wellbeing of the pet 104 at all times. Thus, the smart collar device 100 communicates with a pet owner's smart watch or smart phone 206 using an app 224 to send food and medication reminders. Accordingly, the smart collar device 100 connects to an app 224 to provide pet owners with data and reminders, as needed. Specifically, the smart collar device 100 allows the pet owner to easily activate and deactivate reminders for themselves for various activities, such as but not limited to, food and medication reminders, scheduling veterinary appointments, and/or brushing their pet's teeth, etc. Similarly, the app 224 will allow the pet owner to record instances of performing any of these or similar actions, as well.

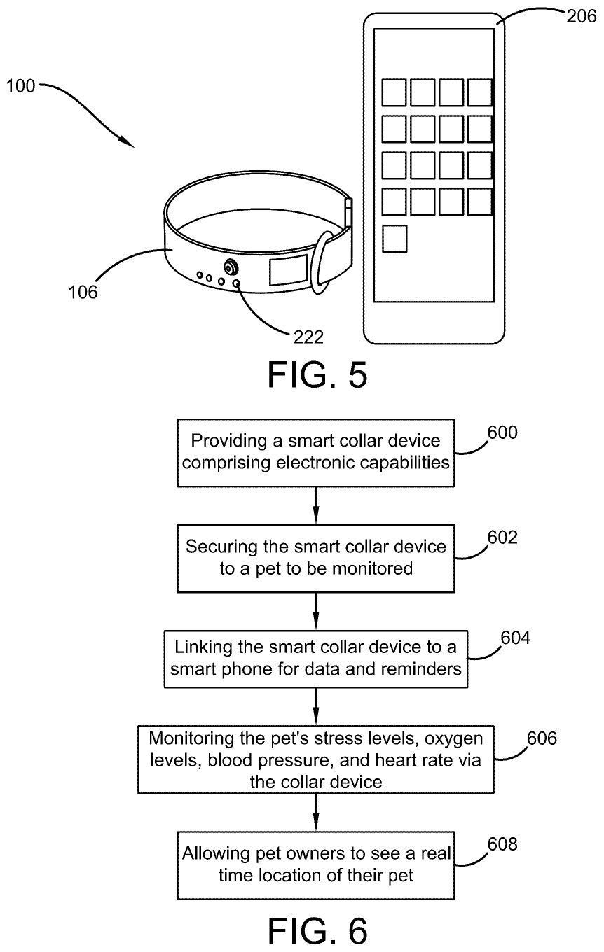

A shown in FIG. 5, the smart collar device 100 is available in a wide variety of colors, designs, patterns, and sizes.

FIG. 6 illustrates a flowchart of the method of monitoring the wellbeing of a pet via their collar. The method includes the steps of at 600, providing a smart collar device comprising electronic capabilities. The method also comprises at 602, securing the smart collar device to a pet to be monitored. Further, the method comprises at 604, linking the smart collar device to a smart phone for data and reminders. The method also comprises at 606, monitoring the pet's stress levels, oxygen levels, blood pressure, and heart rate via the smart collar device. Finally, the method comprises at 608, allowing pet owners to see a real time location of their pet.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “smart collar device”, “smart device”, “collar device”, and “device” are interchangeable and refer to the smart collar device 100 of the present invention.

Notwithstanding the forgoing, the smart collar device 100 of the present invention can be of any suitable size and configuration as is known in the art without affecting the overall concept of the invention, provided that it accomplishes the above stated objectives. One of ordinary skill in the art will appreciate that the smart collar device 100 as shown in FIGS. 1-6 is for illustrative purposes only, and that many other sizes and shapes of the smart collar device 100 are well within the scope of the present disclosure. Although the dimensions of the smart collar device 100 are important design parameters for user convenience, the smart collar device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A smart collar device that provides a user with a means for monitoring the wellbeing of a pet, the smart collar device comprising:
a housing;
a plurality of electronic components for monitoring a pet;
a wireless communication module for establishing a wireless communication channel with a remote electronic device; and
a control button and indicator lights are disposed on the housing to activate or deactivate components of the housing; and
wherein the plurality of electronic components, the control button and indicator lights are disposed on the housing;
wherein the plurality of electronic components monitor stress levels, oxygen levels, blood pressure, and heart rate of the pet; and
further wherein monitored data, reminders, captured images and current location of the smart collar device are transmitted to the remote electronic device using the wireless communication channel.

2. The smart collar device of claim 1 further comprising a band for wrapping around a neck of a pet.

3. The smart collar device of claim 2, wherein the smart collar device is secured around the neck of a pet via a fastening mechanism, such as a clip, a hook and loop, Velcro®, buckle, or snap.

4. The smart collar device of claim 3, wherein the housing is attached to the band or directly integrated within the band.

5. The smart collar device of claim 4 further comprising an LED display for displaying data.

6. The smart collar device of claim 5, wherein the housing comprises a GPS tracker and a camera which allows pet owners to see a real time location of their pet.

7. The smart collar device of claim 6, wherein the band comprises a plurality of LED lights.

8. The smart collar device of claim 7, wherein the band comprises a plurality of reflective strips.

9. The smart collar device of claim 8, wherein the housing is integrally formed into the band or can be utilized as an aftermarket device and secured to a standard pet collar.

10. The smart collar device of claim 9, wherein the smart collar device is water, dust resistant and waterproof.

11. The smart collar device of claim 10, wherein the housing comprises an internal battery.

12. The smart collar device of claim 11, wherein the housing comprises a USB charging port.

13. The smart collar device of claim 12 further comprising a plurality of indicia.

14. A smart collar device that provides a user with a means for monitoring the wellbeing of a pet, the smart collar device comprising:
a band for wrapping around a neck of a pet;
a housing;
a plurality of electronic components for monitoring a pet;
a wireless communication module for establishing a wireless communication channel with a remote electronic device; and
a control button and indicator lights are disposed on the housing to activate or deactivate components of the housing; and
wherein the plurality of electronic components, the control button and indicator lights are disposed on the housing;
wherein the band includes an LED display for displaying data;
wherein the band includes a GPS tracker and a camera which allows pet owners to see a real time location of their pet;
wherein the housing comprises an internal battery and USB charge port;
wherein the plurality of electronic components monitor stress levels, oxygen levels, blood pressure, and heart rate of the pet; and
further wherein monitored data, reminders, captured images and current location of the smart collar device are transmitted to the remote electronic device using the wireless communication channel.

15. The smart collar device of claim 14, wherein the housing is attached to the band or directly integrated within the band.

16. The smart collar device of claim 14, wherein the remote electronic device is a smart phone, smart device, smart pad, or personal digital assistant.

17. The smart collar device of claim 14, wherein the wireless communication module is in form of an integrated circuit or a microchip antenna and is configured to provide Bluetooth/Wi-Fi capabilities.

18. The smart collar device of claim 14, wherein the remote electronic device comprises an installed software application that receives information related to the pet.

19. The smart collar device of claim 14, wherein the smart collar device is available in a variety of colors, designs, patterns, and sizes.

20. A method of monitoring the wellbeing of a pet via their collar, the method comprising the following steps:
providing a smart collar device comprising electronic capabilities;
securing the smart collar device to a pet to be monitored;
linking the smart collar device to a smart phone for data and reminders;
monitoring the pet's stress levels, oxygen levels, blood pressure, and heart rate via the smart collar device; and
allowing pet owners to see a real time location of their pet.
